(19) United States
(12) Patent Application Publication
(10) Pub. No.: US 2009/0012150 A1
(43) Pub. Date: Jan. 8, 2009

(54) Stablized Pharmaceutical Composition Comprising an Amorphous Active Substance

(76) Inventors: Rok Grahek, Kranj (SI); Andrej Bastarda, Vrhnika (SI); Mateja Salobir, Ljubljana (SI)

Correspondence Address: DARBY & DARBY P.C., P.O. BOX 770, Church Street Station, New York, NY, 10008-0770, US

(21) Appl. No.: 11/923,059

(22) Filed: Oct. 24, 2007

Related U.S. Application Data
(63) Continuation of application No. 10/683,683, filed on Oct. 10, 2003, now abandoned.

(30) Foreign Application Priority Data
Oct. 11, 2002 (SI) P200200244

(51) Int. Cl. A61K 31/40

(52) U.S. Cl. 514/423

(57) ABSTRACT

The invention relates to the pharmaceutical composition comprising the amorphous active substance which is atorvastatin calcium. The process of stabilization of the pharmaceutical composition comprising the pharmaceutical formulation with amorphous atorvastatin calcium, the process of stabilization of the pharmaceutical formulation comprising amorphous atorvastatin calcium and the process of stabilization of atorvastatin calcium in an amorphous form is described.

### FIELD OF THE INVENTION

The present invention belongs to the field of pharmaceutical technology and relates to the pharmaceutical composition comprising the amorphous active substance which is atorvastatin calcium. The active substance is useful for treating of hypercholesterolemia and hyperlipidemia. The invention enables the preparation of a stable pharmaceutical composition comprising the amorphous active substance, known to be unstable in an acidic environment and susceptible to heat, light, moisture and low pH, in a technologically simple way.

### PRIOR ART

Atorvastatin calcium, the substance which is known under the name (R—(R*, R*))-2-(4-fluorophenyl)-β,δ-dihydroxy-5-(1-methylethyl)-3-phenyl-4((phenylamino)carbonyl)-1H-pyrrole-1-heptanoic acid hemi calcium salt is useful as an inhibitor of 3-hydroxy-3-methylglutaryl-coenzyme A reductase (HMG-CoA reductase), an enzyme catalyzing the intracellular synthesis of cholesterol. Therefore, HMG-CoA reductase enzyme inhibitors are considered especially useful in the treatment of hypercholesterolemia and hyperlipidemia.

The processes for the preparation of atorvastatin calcium and key intermediates thereof are described in U.S. Pat. Nos. 5,003,080; 5,097,045; 5,103,024: 5,124,482; 5,149,837; 5,155,251; 5,216,174; 5,245,047; 5,248,793; 5,280,126; 5,342,952; and 5,397,792.

Atorvastatin calcium can exist in an amorphous form or in different crystalline forms which are disclosed in the patent applications WO 97/3958; WO 97/3959; WO 01/36384; WO 02/41834; WO 02/43732; WO 02/51804, and WO 02/57229. The processes for the preparation of amorphous atorvastatin calcium are described in the patent applications WO 97/3960; WO 00/71116; WO 01/28999; WO 01/42209; WO 02/57228; and WO 02/59087.

It is well known that active substances in an amorphous form are better soluble and dissolve more rapidly, respectively, than in a crystalline form. The advantage of an amorphous active substance over a crystalline form is particularly evident in case of less soluble substances such as, for example, atorvastatin calcium, and it is manifested in better bioavailability of an active substance.

It is known from the patent and relevant literature that atorvastatin calcium is an unstable substance which is susceptible to heat, moisture, light and low pH at which atorvastatin calcium is converted from the carboxylic acid form to the lactone form (U.S. Pat. No. 5,686,104; Hurley, T. R. et al Tetrahedron (1993). 49, 1979-1984). The problem of instability of atorvastatin calcium has been solved thus far by the addition of excipients to a pharmaceutical formulation with special emphasis to stabilization of atorvastatin calcium in the sense of conversion into the lactone form by the addition of a basifying or a buffering agent to a pharmaceutical composition (WO 00/35425; WO 94/16603). A procedure for stabilization of an active substance is known when in the final phase of synthesis an alkaline substance or a buffering solution is added to prepare an alkaline stabilized substance as described in the patent application WO 01/93860.

The use of a pharmaceutical formulation comprising amorphous atorvastatin calcium as the active substance is advantageous over a pharmaceutical formulation comprising a crystalline substance because the amorphous substance dissolves faster and better which is an important factor for bioavailability of the active substance in the body. It is well known that the stability of an active substance depends on a polymorphous form in which it exists and that an amorphous form is less stable than a crystalline form indicating that an amorphous form compared to a crystalline form is even more susceptible to heat, light, moisture and low pH. All these factors are of key importance for the stability of a pharmaceutical formulation comprising an amorphous substance. Impurities generated at degradation of an active substance reduce a therapeutic effect of an active substance and additionally unnecessarily burden the body with unnecessary degradation products. To date an appropriate and useful pharmaceutical composition containing comprising atorvastatin calcium has not been described so far.

Therefore, there is a constant need for preparing a stable pharmaceutical composition comprising amorphous atorvastatin calcium. The principal object of the present invention is the preparation of a pharmaceutical composition comprising amorphous substance atorvastatin calcium being advantageous over a crystalline substance by better bioavailability and that is prepared according to the process which is simple and economically convenient.

### DESCRIPTION OF THE INVENTION

We have surprisingly found that the stability of amorphous atorvastatin calcium is affected by oxygen content in an environment in which there is an amorphous substance or a pharmaceutical formulation comprising an amorphous substance or a pharmaceutical composition comprising an amorphous substance. There is a linear dependence between the assay of degradation products and the oxygen content in atmosphere. If half of the oxygen content in e atmosphere is replaced with an inert gas, generation of degradation products is halved over a fixed period of time under defined temperature conditions. If amorphous atorvastatin calcium is stored at a defined temperature in an atmosphere with minimal oxygen content, after a certain period of time the assay of degradation products is less than and/or equal to that when crystalline atorvastatin calcium is stored in air.

The basic objective of the present invention is to prepare a stable pharmaceutical formulation comprising an active substance which is amorphous atorvastatin calcium. This aim is achieved by the procedure of packaging a pharmaceutical formulation into for gas exchange non-permeable package in an inert gas atmosphere which means exchange and expelling way comprising as the active substance an amorphous substance known from the literature to be less stable than a crystalline from, appears to have unexpectedly superior and/or equal stability to a pharmaceutical composition comprising a crystalline form of the same substance if packaging is carried out in air. The process of improving the stability of a pharmaceutical composition comprising amorphous atorvastatin calcium in an inert gas atmosphere during the packaging procedure of the pharmaceutical formulation into for gas exchange n on-permeable package such as, for example, Al/Al blister, Al-polychloro-3-fluoroethylene homopolymer/PVC laminate blister and bottles, which is the object of the present invention, is great advantage over to date known processes of improving stability of an amorphous active substance because the process is technologically simple and economically non-demanding; at the same time, a stabilized pharmaceutical composition comprising amorphous atorvastatin calcium, prepared by said process, does not burden the body with additional substances. Additional great priority of the method for stabilization of the pharmaceutical product of the present invention is that the attained stability of the pharmaceutical composition is superior to that with crystalline atorvastatin calcium.

The pharmaceutical composition of the present invention is the pharmaceutical formulation comprising amorphous atorvastatin calcium as the active substance and pharmaceutically acceptable excipients. The pharmaceutical formulation may be in any form such as, for example, tablets, orally dispersible pharmaceutical formulations, capsules, pellets, granulate, etc., suitable to be stored in for gas exchange non-permeable package. Nitrogen or argon can be used as inert gas atmosphere in the packaging procedure.

The stable pharmaceutical product of the present invention is used in the treatment of hypercholesterolemia and hyperlipidemia.

The object of the present invention is also the process for preparation of the stable pharmaceutical formulation comprising as the active substance atorvastatin calcium in an amorphous form and pharmaceutically acceptable excipients. This aim is achieved by storing the pharmaceutical formulation in an inert atmosphere thereby achieving the stability which is superior and/or equal to the stability of the pharmaceutical formulation comprising the crystalline active substance. The process of stability improvement of the pharmaceutical formulation comprising amorphous atorvastatin calcium by storing the pharmaceutical formulation in an inert atmosphere, which is the object of the present invention, is a great advantage over so far known processes of stability improvement of the amorphous active substance, as the process is technologically simple and economically non-demanding; furthermore, the pharmaceutical formulation, prepared by this process, does not burden the body with additional substances. The term inert atmosphere may be understood to mean the atmosphere with the minimal oxygen content.

Analyzing the assay of degradation products in the pharmaceutical formulation comprising amorphous atorvastatin calcium we have found that the assay of degradation products in the amorphous substance strongly increases when stored in air. We have surprisingly found that the stability of the pharmaceutical formulation is significantly improved if the pharmaceutical formulation is stored in an atmosphere with the minimal oxygen content. If a pharmaceutical formulation comprising amorphous atorvastatin calcium is stored at a defined temperature in an atmosphere with the minimal oxygen content, over a certain period of time the assay of degradation products is less than and/or equal to that in a pharmaceutical formulation comprising crystalline atorvastatin calcium stored in air.

The pharmaceutical formulation of the present invention comprises an amorphous form of atorvastatin calcium as the active substance and pharmaceutically acceptable excipients. The pharmaceutical formulation of the present invention can be any form that is used in pharmaceutical industry such as, for example, tablets, orally dispersible formulations, capsules, pellets, granulate, etc. Nitrogen or argon can be used as the inert gas for maintenance of an inert atmosphere. The pharmaceutical formulation can be stored in an inert atmosphere in Al/Al blister, Al-polychloro-3-fluoroethylene homopolymer/PVC laminate blister or bottles.

The pharmaceutical formulation of the present invention is useful in the treatment of hypercholesterolemia and hyperlipidemia.

The further object of the present invention is also improvement of the stability of the active substance atorvastatin calcium in an amorphous form. The aim is achieved by storing the active substance in an inert atmosphere which from the technological and economic point is considered to be an extremely simple solution. Stability of the amorphous active substance stored in this manner is equivalent to the stability of crystalline atorvastatin calcium. The active substance is stored in practically for gas exchange non-permeable packaging such as, for example, metal containers, glass containers, for gas non-permeable plastic bags or containers. Nitrogen or argon can be used as an inert gas for maintenance of an inert atmosphere.

The stable amorphous active substance of the present invention is useful in the treatment of hypercholesterolemia and hyperlipidemia.

The present invention is illustrated but in no way limited by the following examples:

### EXAMPLE 1

Samples each containing 100 mg of crystalline and amorphous atorvastatin calcium were transferred into 10-ml rubber-stoppered vials, in an atmosphere with different oxygen content in reference to air composition. Nitrogen of 99% purity (vol/vol) purity was used as an inert gas. The samples were stored at 80° C. for 6 days, and then the assay of impurities was determined by High Performance Liquid Chromatography.

The oxygen content in the vials was determined by gas chromatography with a mass spectrometric detector (GC/MS).

The degradation products were determined by gas chromatography Agilent Technologies (Waldbronn, Germany) model HP 1100. Chromatograms were recorded by a UV detector at 250 nm. The column Chromolit Performance RP-18e 100×4.6 mm (Merck, Darmstadt, Germany) and the gradient of mobile phase A: 20 mM ammonium acetate pH 4.0, 5% (v/v) tetrahydrofuran and 25% (v/v) acetonitrile and the gradient of mobile phase B: 20 mM ammonium acetate pH 4.0, 5% (v/v) tetrahydrofuran and 70 (v/v) acetonitrile were used. The composition of the mobile phase was changed so that initially and to minute 2 there was 25% of mobile phase B (75% of mobile phase A), from minute 2 to minute 4.7 the composition of the mobile phase changed linearly to 100% mobile phase B and it remained such to the end of the analysis at minute 5.5. Flow rate of the mobile phase was 7 ml/min. Under the listed conditions the retention time of atorvastatin calcium is approximately 1.4 minute. The samples of atorvastatin calcium were dissolved in the solvent 200 mM Tris p H 7, 40% (v/v) acetonitrile of the concentration 0.5 mg/ml.

Analytical method for the determination of the assay of oxygen in the vials and blisters:

The analyses were performed on a gas chromatograph Varian (Walnut Creek, USA) model Varian 3400 and mass detector Finnigan (San Jose, USA) model SSQ700. The capillary column PLOT Coating Molsieve 5A 25 m long of internal diameter 0.32 mm (Variana) was used. Temperature of the column was 50° C., the injector 150° C. and the connection with the mass detector 150° C. Flow through the column was 2 ml of helium per minute. Mode of injection was split with the 1:100 ratio. The mass detector operated by ionization with electrons at 70 eV within the mass region from 20 to 60 mass units in 0.3 second.

Vials and blisters were in the chamber with argon atmosphere. 50 μl gas samples were taken by a gas injection and immediately analyzed. Argon atmosphere prevented contamination of the samples with oxygen during sampling.

The signal of oxygen molecular ion 32 m/z with retention approx. 1.9 minutes and the signal of nitrogen molecular ion 28 m/z with retention approx. 3.1 minutes was detected by the gas detector. The result was calculated to absent oxygen content in reference to the sum of oxygen and nitrogen.

TABLE 1
Increase of the assay of degradation products in the substances
stored under stress conditions (6 days at temperature 80° C.)
in comparison with the starting active substance

| | Crystalline | Amorphous |
|---|---|---|
| | atorvastatin calcium | atorvastatin calcium |
| Oxygen | Increase in the assay of | Increase in the assay of |
| content in | degradation products in | degradation products in |
| atmosphere | % in reference to starting | % in reference to starting |
| in % | active substance | active substance |
| 0.6 | 0 | 0 |
| 2.8 | 0 | 0.13 |
| 12.2 | 0.02 | 0.49 |
| 21.3 | 0.05 | 0.89 |

The testing results of the assay of degradation products in crystalline and amorphous atorvastatin calcium at different oxygen contents in atmosphere have demonstrated that at minimal oxygen content the assay of degradation products is equal in both active substances indicating that the stability of amorphous atorvastatin calcium stored in an inert atmosphere is equal to the stability of crystalline atorvastatin calcium. The measurements also show that the procedure of storing amorphous atorvastatin calcium in an inert atmosphere improves the stability of the amorphous substance but it has no influence on the stability of crystalline atorvastatin calcium.

### EXAMPLE 2

Tablets containing 10 mg of amorphous atorvastatin calcium, not previously stabilized by storing in an inert atmosphere, and the other pharmaceutically acceptable excipients (microcrystalline cellulose, lactose monohydrate, crosslinked carboxymethyl cellulose, polysorbate 80, hydroxypropyl cellulose, magnesium oxide) were stored in glass 10-ml rubber-stoppered vials in normal atmosphere (air) and in atmosphere with different oxygen content which was replaced by inert gas. Nitrogen of 99% purity (vol/vol) was used as an inert gas. For comparison, tablets containing 10 mg of crystalline atorvastatin calcium stored in 10-ml rubber-stoppered vials in normal atmosphere (air) were used. Each vial contained one tablet. These vials were placed in a drier for six days under stress conditions, that is, at 80° C.

The samples for analysis of the assay of degradation products were prepared by adding 10 ml of a solvent to the tablet in a suitable container and dissolving the tablet by ultrasound in an ultrasound bath for 10 minutes. The tablet disintegrated and the resulting suspension was filtered through the PTFE 0.45 μm injection filter. The clear solution was analyzed by the method disclosed in Example 1.

TABLE 2
Increase of the assay of degradation products in comparison with the
starting active substance - amorphous atorvastatin calcium in the tablets
stored under the stress conditions (6 days at 80° C.)

| Tablet stored in an | Increase in the assay of degradation |
|---|---|
| atmosphere with oxygen | products in % in reference to starting active |
| content in % | substance |
| 21.0 | 3.11 |
| 12.4 | 0.94 |
| 0.4 | 0.01 |

TABLE 3
Increase of the assay of degradation products in comparison with the
starting active substance - crystalline atorvastatin calcium in the
tablets stored under the stress conditions (6 days at 80° C.)

| Tablet stored in an | Increase in the assay of degradation |
|---|---|
| atmosphere with oxygen | products in % in reference to starting active |
| content in % | substance |
| 21.2 | 0.30 |

Testing results of the assay of degradation products in the pharmaceutical formulation comprising amorphous atorvastatin calcium stored in an atmosphere with different oxygen content have demonstrated that at the minimal oxygen content in atmosphere the increase of the assay of degradation products is within the analysis error. The stability of the pharmaceutical formulation comprising amorphous atorvastatin calcium stored in an inert atmosphere, is superior to the stability of a pharmaceutical formulation comprising crystalline atorvastatin calcium, store in normal atmosphere. It is opposite when the pharmaceutical formulation comprising amorphous atorvastatin is stored in normal atmosphere and the stability of the active substance is considerably lower because there is an increase of only 3% of the assay degradation product assay to the initial value of amorphous substance atorvastatin calcium. It is interesting that already with half-replacing oxygen with an inert gas, the increase drops for two-thirds relative to the values when a pharmaceutical formulation is stored in air.

### EXAMPLE 3

Tablets containing 20 mg of amorphous atorvastatin calcium, not previously stabilized by storing in an inert atmosphere, and the other pharmaceutically acceptable excipients (microcrystalline cellulose, lactose monohydrate, crosslinked carboxymethyl cellulose, polysorbate 80, hydroxypropyl cellulose, magnesium oxide) were packed into blisters with aluminum foil on an industrial blister packaging machine. The first tablet batch was packed in normal atmosphere (air). The second batch, prior to upper foil sealing, packed in an atmosphere of technical argon 99% (v/v). For comparison, the tablets containing 10 mg of crystalline atorvastatin in normal atmosphere were stored in 10-ml vials. The oxygen content in the blister in argon atmosphere was determined by gas chromatography with a mass spectrometric detector (GS/MS).

The stress test of storing the blisters under stress conditions (days at 80° C.) was carried out. For comparison, the tablets containing 10 mg of crystalline atorvastatin calcium in blisters with in normal atmosphere (air) were used and they were exposed to the stress condition. The difference in the assay of impurities of atorvastatin in the blisters was determined by the method described in Example 2.

TABLE 4
Increase of the assay of degradation products in comparison
with the starting active substance - amorphous/crystalline
atorvastatin calcium in the tablets in dependence upon
the oxygen content in atmosphere at packaging

| | | Amorphous | Crystalline |
|---|---|---|---|
| | | substance | substance |
| | | Increase of the | Increase of the |
| | Oxygen | assay of degradation | assay of degradation |
| | content | products in % to | products in % to |
| | in % in | starting active | starting active |
| Sample | atmosphere | substance | substance |
| Normal | | | |
| atmosphere | | | |
| (air) | | | |
| Tablet 1 | 21.0 | 0.91 | 0.13 |
| Tablet 2 | 21.0 | 0.87 | 0.15 |
| Tablet 3 | 21.0 | 0.95 | 0.12 |
| Inert gas | | | |
| atmosphere | | | |
| Tablet 1 | 2.4 | 0.01 | |
| Tablet 2 | 2.3 | 0.02 | |
| Tablet 3 | 2.1 | 0.03 | |

Testing results of the assay of degradation products in the pharmaceutical composition comprising amorphous atorvastatin calcium when the pharmaceutical formulations were packed into the blisters in inert gas atmosphere indicate that an increase of the assay of degradation products under stress conditions in the amorphous active substance is within the analysis error suggesting that the amorphous substance, pharmaceutical formulation or pharmaceutical composition, respectively, comprising the amorphous substance, is stable during prolonged storage if an inert gas atmosphere is used during packaging into blisters. The results surprisingly indicate the fact that the method of stabilization of the pharmaceutical composition comprising amorphous atorvastatin calcium and comprising packaging of the pharmaceutical formulation into a blister in an inert gas atmosphere provides better results in the stability than the pharmaceutical composition comprising crystalline atorvastatin calcium packed in air.

1. A stable pharmaceutical composition which comprises a pharmaceutical formulation with an amorphous active substance wherein the pharmaceutical formulation is packed into for gas exchange non-permeable package with packaging carried out in an inert gas atmosphere.

2. The stable pharmaceutical composition according to claim 1 wherein the amorphous active substance is atorvastatin calcium.

3. The stable pharmaceutical composition according to claim 1 wherein for gas exchange non-permeable package is Al/Al blister, Al-polychloro-3-fluoroethylene homopolymer/PVC laminate blister or bottle.

4. The stable pharmaceutical composition according to claim 3 wherein for gas exchange non-permeable package is the Al/Al blister.

5. The stable pharmaceutical composition according to claim 1 wherein an inert gas is nitrogen or argon.

6. The stable pharmaceutical composition according to claim 5 wherein the inert gas is nitrogen.

7. The stable pharmaceutical composition according to Claim 1 wherein the pharmaceutical composition is prepared in the form of tablets, o rally dispersible pharmaceutical formulations, capsules, pellets or granulate.

8. A stable pharmaceutical formulation comprising an amorphous active substance and pharmaceutically acceptable excipients which is stored in an inert atmosphere.

9. The stable pharmaceutical formulation according to claim 8 wherein the amorphous active substance is atorvastatin calcium.

10. The stable pharmaceutical formulation according to claim 8 wherein the gas for maintenance of an inert atmosphere is nitrogen or argon.

11. The stable pharmaceutical formulation according to claim 10 wherein the gas for maintenance of an inert atmosphere is nitrogen.

12. The stable pharmaceutical formulation according to claim 8 which is prepared in the form of tablets, orally dispersible pharmaceutical formulations, capsules, pellets or granulate.

13. The stable pharmaceutical formulation according to claim 8 which is stored into for gas non-permeable package such as Al/Al blister, Al-polychloro-3-fluoroethylene homopolymer/PVC laminate blister or bottle.

14. The stable pharmaceutical formulation according to claim 13 wherein for gas non-permeable package is the Al/Al blister.

15. A stable amorphous active substance which is stored in an inert atmosphere.

16. The stable amorphous substance according to claim 15 wherein the amorphous active substance is atorvastatin calcium.

17. The stable amorphous active substance according to claim 15 wherein the gas for maintenance of an inert atmosphere is nitrogen or argon.

18. The stable amorphous active substance, according to claim 15 wherein the gas for maintenance of an inert atmosphere is nitrogen.

19. The stable amorphous active substance, according to claim 15 wherein the active substance is stored into for gas exchange non-permeable packaging such as a metal container, glass container, for gas non-permeable plastic bag or for gas non-permeable plastic container.

20. The stable amorphous active substance according to Clam 19 wherein for the exchange non-permeable packaging is the plastic bag.

21. A method of stabilization of the pharmaceutical composition comprising the pharmaceutical formulation with the amorphous active substance wherein the pharmaceutical formulation is packed into for gas exchange non-permeable packaging and the packaging procedure is carried out in an inert gas atmosphere.

22. The method of stabilization of the pharmaceutical composition according to claim 21 wherein the amorphous active substance is atorvastatin calcium.

23. The method of stabilization of the pharmaceutical composition according to claim 21 wherein for gas exchange non-permeable packaging is Al/Al blister, Al-polychloro-3-fluoroethylene homopolymer/PVC laminate blister or bottle.

24. The method of stabilization of the pharmaceutical composition according to claim 23 wherein for gas exchange non-permeable packaging is the Al/Al blister.

25. The method of stabilization of the pharmaceutical composition according to claim 21 wherein an inert gas is nitrogen or argon.

26. The method of stabilization of the pharmaceutical composition according to claim 25 wherein the inert gas is nitrogen.

27. The method of stabilization of the pharmaceutical composition according to claim 21 wherein the pharmaceutical formulation is prepared in the form of tablets, orally dispersible pharmaceutical formulations, capsules, pellets pr granulate.

28. A method of stabilization of a pharmaceutical formulation comprising an amorphous active substance and pharmaceutically acceptable excipients wherein a pharmaceutical formulation is stored in an inert atmosphere.

29. The method of stabilization of the pharmaceutical formulation according to claim 28 wherein the amorphous active substance is atorvastatin calcium.

30. The method of stabilization of the pharmaceutical formulation according to claim 28 wherein the gas for maintenance of an inert atmosphere is nitrogen or argon.

31. The method of stabilization of the pharmaceutical formulation according to claim 30 wherein the gas for maintenance of an inert atmosphere is nitrogen.

32. The method of stabilization of the pharmaceutical formulation according to claim 28 which is packed into for gas non-permeable packaging such as Al/Al blister, Al-polychloro-3-fluoroethylene homopolymer/PVC laminate blister or bottle.

33. The method of stabilization of the pharmaceutical formulation according to claim 32 wherein for gas non-permeable packaging is the Al/Al blister.

34. The method of stabilization of the pharmaceutical formulation according to claim 28 which is prepared in the form of tablets, orally dispersible pharmaceutical formulations, capsules, pellets or granulate.

35. A method of stabilization of an amorphous active substance wherein an amorphous substance is stored in an inert atmosphere.

36. The method of stabilization of the amorphous active substance according to claim 35 wherein the amorphous active substance is atorvastatin calcium.

37. The method of stabilization of the amorphous active substance according to claim 35 wherein the gas for maintenance of an inert atmosphere is nitrogen or argon.

38. The method of stabilization of the amorphous active substance according to claim 37 wherein the gas for maintenance of an inert atmosphere is nitrogen.

39. The method of stabilization of the amorphous active substance according to claim 35 wherein the active substance is packed into for gas non-permeable packaging such as metal container, glass container, for gas non-permeable plastic bag or for gas non-permeable plastic container.

40. The method of stabilization of the amorphous active substance according to claim 39 wherein for gas exchange non-permeable packaging is for gas non-permeable plastic bag.

41. The stable pharmaceutical composition comprising the pharmaceutical formulation with the amorphous active substance which is prepared by the process according to claims 21 to 27.

42. The stable pharmaceutical formulation comprising the amorphous active substance and pharmaceutically acceptable excipients which is prepared by the process according to claims 28 to 34.

43. The stable amorphous active substance which is prepared by the process according to claims 35 to 40.

44. The stable pharmaceutical composition according to claim 1 is useful in treatment of hypercholesterolemia and hyperlipidemia.

45. The stable pharmaceutical formulation according to claim 8 is useful in the treatment of hypercholesterolemia and hyperlipidemia.

46. The stable amorphous active substance according to claim 15 is useful in the treatment of hypercholesterolemia and hyperlipidemia.
